(19) United States
(12) Patent Application Publication
(10) Pub. No.: US 2003/0225169 A1
(43) Pub. Date: Dec. 4, 2003

(54) Portable gas-to-liquids unit and method for capturing natural gas at remote locations

(75) Inventor: Richard D. Yetman, Tomball, TX (US)

Correspondence Address: GARY L. BUSH, ANDREWS & KURTH, L.L.P., SUITE 4200, 600 TRAVIS, HOUSTON, TX, 77002, US

(73) Assignees: GLT Microsystems AG; GTL Microsystems AG

(21) Appl. No.: 10/446,377

(22) Filed: May 28, 2003

Related U.S. Application Data
(60) Provisional application No. 60/384,072, filed on May 28, 2002.

(51) Int. Cl. B01J 8/04; C07C 27/26

(52) U.S. Cl. 518/726; 422/190

(57) ABSTRACT

A method and apparatus for converting natural gas from a remote source into hydrocarbon liquid stable at room temperature, comprising a skid or trailer-mounted portable gas-to-liquids reactor. The reactor includes a preprocessor which desulfurizes and dehydrates the natural gas, a first-stage reactor which transforms the preprocessed natural gas into synthesis gas, and a liquid productions unit using a Fisher-Tropsch or similar polymerization process. The hydrocarbon liquid may be stored in a portable tank for later transportation or further processed on site in a portable hydrocarbon cracking unit to yield fuel or lubricating oils.

### CROSS REFERENCE TO RELATED APPLICATION

This application is based upon provisional application 60/384,072, filed on May 28, 2002 the priority of which is claimed.

### BACKGROUND OF THE INVENTION

1. Field of the Invention

This invention relates generally to a method and apparatus arranged and designed for converting natural gas at a remote land location to a non-cryogenic liquid for storage and transport by land vehicle to another location or for conversion to a motor fuel on site.

2. Description of the Prior Art

A large number of gas fields on land are “stranded fields”, meaning that they are not close enough to a pipeline to be economically feasible for production. As a result, such fields are not developed and the economic value of the gas remains trapped in the earth's crust.

Oil wells on the other hand can be developed even if such wells are in a remote location, because liquid crude oil can be collected in a tank at a remote well and then transferred to a refinery by a tanker truck.

In some cases, natural gas may be available at a remote location, say in a pipeline. However, such natural gas has greater utility if converted in situ to a liquid motor fuel.

Gas-to-liquids (GTL) technology for converting natural gas, which consists primarily of methane, has existed for more than half a century, but a recent resurgence of interest is providing significant advancements in the rapidly growing art. Prior art teaches that natural gas may be converted to higher molecular weight hydrocarbons by generally two techniques—either a direct transformation or a transformation with an intermittent step of creating a synthesis gas (syngas), a gas composed generally of hydrogen and carbon monoxide.

Direct transformation into higher molecular weight hydrocarbons may occur through Pyrolysis, during which methane at generally 250° C. to 1700° C. is passed through a catalyst in the absence of substantial amounts of oxygen. Processes and catalysts are described in U.S. Pat. Nos.: 4,199,533; 4,547,607; 4,704,496; 4,801,762; 5,093,542; 5,157,189; and 5,245,124. These processes require high activation energy and can be difficult to control. As a result, there is minimal commercial use of direct GTL processes.

Two or three stage GTL processes, where the natural gas is first converted to syngas, have more prevalent commercial use than direct processes. For example, Mobil has developed M-Gasoline, which is created by a three-stage process. Natural gas is converted to syngas, which is then transformed methanol, which is finally made into M-gasoline. However, the most common GTL process is a two stage process in which the natural gas is first converted to syngas, which is then changed into a liquid hydrocarbon via the Fisher-Tropsch (F-T) process.

In the first step of the two-stage GTL process, conversion of natural gas to syngas is achieved by steam reforming, partial oxidation, or a combination of both. Steam reforming, performed in a heater with catalyst-filled tubes, is endothermic and produces syngas in a 3:1 hydrogen to carbon monoxide ratio. Because the subsequent F-T process requires a 2:1 stoichiometric ratio, steam reforming results in excess hydrogen production, which may be useful as feedstock for other manufacturing processes. On the other hand, partial oxidation produces a 2:1 stoichiometric ratio, but it requires a source of oxygen. A pure oxygen source produces a pure synthesis gas, but an air-based process, which produces synthesis gas diluted with nitrogen, reduces the need for costly oxygen plants. The partial oxidation process is highly exothermic.

Next, the synthesis gas is polymerized via the F-T process to form a synthetic crude (syncrude). The reaction occurs on the surface of an iron-based or cobalt-based heterogeneous catalyst in either a vertical tube reactor or a slurry reactor. The resultant product at room temperature ranges from a solid or waxy substance to a liquid, depending on the temperature and pressure maintained during the reaction. Since the F-T process is also highly exothermic, the reactor vessels require cooling; steam is generally a byproduct.

A low-cost GTL plant is described in a paper presented at the 1998 Offshore Technology Conference in Houston, Tex., the contents of which are incorporated herein by reference and made a part hereof. Dr. David D. J. Anita and Dr. Duncan Seddon, OTC 8901 Low Cost 10MMCF/D Gas to Syncrude Plant for Associated Gas, 30^{th} Annual Offshore Technology Conference 1998 Proceedings, Volume 4, 753.

Identification of Objects of the Invention

A primary object of the invention is to provide a method and apparatus for converting natural gas at a remote location to a hydrocarbon characterized by having a liquid phase at ambient air temperature and atmospheric pressure, hereinafter simply referred to as liquid syncrude, for refining on site or for transportation to a distant refinery.

Another object of the invention is to provide a trailer-mounted or palletized GTL unit at a remote source of natural gas such as a gas well, for converting the natural gas to liquid syncrude which can be stored in a fixed tank or a tanker truck.

Another object of the invention is to provide a trailer-mounted or palletized GTL unit at a remote source of natural gas such as a gas well or a gas pipeline, in combination with a trailer-mounted or palletized hydrocarbon cracking unit for converting natural gas on site to a common motor fuel such as diesel or gasoline.

### SUMMARY OF THE INVENTION

The objects identified above, as well as other features and advantages of the invention are incorporated in an apparatus including a palletized or trailer-mounted GTL unit which converts natural gas to liquid syncrude. The apparatus further includes a palletized or trailer-mounted hydrocracker for converting the liquid syncrude to a common motor fuel such as diesel or gasoline and a tank for collecting the effluent.

The GTL unit comprises a gas preprocessor to filter and condition the incoming natural gas, a syngas reactor which contains catalyst to reform the natural gas forming a syngas, and a Fisher-Tropsch reactor to convert the syngas to liquid syncrude.

The method of the invention includes placing a portable GTL unit next to a land-based source of natural gas, conducting natural gas to the GTL unit, and converting it to liquid syncrude. The method includes collecting the liquid syncrude in a tank and transporting it to a distant refinery. Alternatively, the liquid syncrude is processed by a local hydrocarbon cracking unit creating diesel or gasoline to fuel military or commercial motor vehicles.

### BRIEF DESCRIPTION OF THE DRAWINGS

The invention is described in detail hereinafter on the basis of the embodiments represented schematically in the accompanying figures, in which:

FIG. 1 illustrates a trailer-mounted GTL unit parked in proximity to a gas well with a tanker truck for transporting liquid syncrude to another location.

FIG. 2 illustrates a skid-mounted GTL unit located at a point along a natural gas pipeline, a skid-mounted hydrocarbon cracking unit and a storage tank, for converting natural gas to a ready local source of refined fuel.

### DESCRIPTION OF THE PREFERRED EMBODIMENT OF THE INVENTION

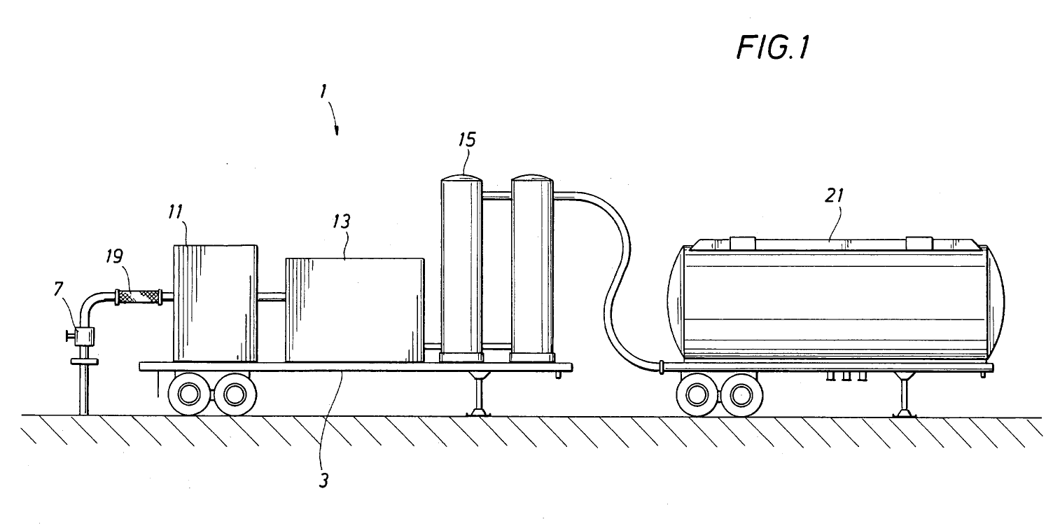

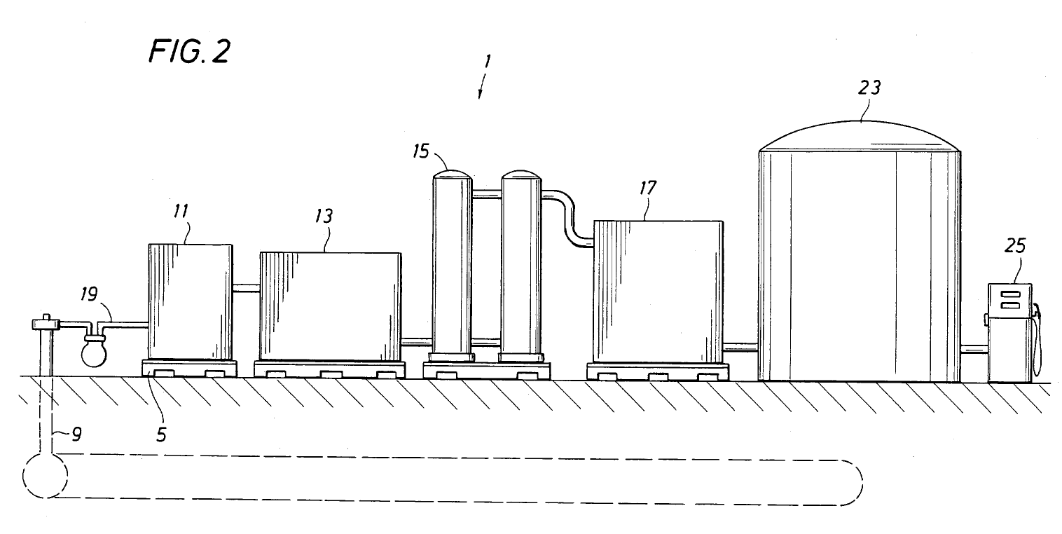

FIGS. 1 and 2 illustrate compact GTL equipment 1 which is arranged and designed to be portable. The term portable is used here to mean that the equipment can be placed on a trailer 3 as illustrated in FIG. 1 or modularly mounted on skids 5 as shown in FIG. 2. Palletized GTL equipment can be readily transported to remote locations by common cargo handling equipment. The GTL equipment converts natural gas from a source, such as a gas well 7 (FIG. 1) or pipeline 9 (FIG. 2), to liquid syncrude for storage and/or refinement.

The portable GTL equipment includes generally a gas preprocessing unit 11, a first stage reactor 13, a second stage reactor 15 (also known as a liquids production unit) and an optional hydrocracker unit 17 (FIG. 2). The hydrocracker unit 17 is not necessary if on-site production of common petrochemicals is not desired. A connector pipe or hose 19 provides a fluid flow path from the gas source 7,9 to the GTL equipment 1. In the preferred embodiment, the first stage reactor is a syngas reactor and the second stage reactor is a F-T reactor, although other methods are within the scope of the invention, including single-stage polymerization.

Syngas and F-T reactors which are commercially in use are generally too large in size for an economical yield to fit on a trailer as illustrated in FIG. 1. The reactors of this invention are smaller in size due to process intensification technologies in which reactors and catalysts are designed and arranged to significantly increase the surface area to volume ratio of catalyst sites. This micro-reactor technology results in small reactors with high gas flow rates. For a given flow rate, a typical reduction in reactor size ranges from one to two orders of magnitudes from those commercially available today.

In the gas preprocessing unit 11, natural gas with potentially wide ranging characteristics is conditioned by filtering, desulphering and dehydrating. The preprocessing unit also provides pressure regulation, flow control and mixture with air for input to the syngas reactor.

The feed gas/steam mixture is converted to syngas in the first-stage 13 or syngas reactor. Although air-fed and oxygen-fed partial oxidation reactions are within the scope of the invention, the preferred process is for a steam methane reforming reaction. In this reaction, the feed gas/steam mixture is introduced into a catalyst at elevated temperature (and possibly pressure). The reforming reaction yields a syngas mixture with a H₂:CO ratio of 3:1. The process intensification catalyst may comprise a metallic substrate with a γ-alumina support and an active promotor metal (such as platinum or rhodium). U.S. patent application 20,020,035,036, which is incorporated herein, describes such a configuration which offers an economical catalyst with high conversion and selectivity. Alternatively, U.S. patent application 20,020,009,407, incorporated herein, describes a catalyst made of an open reticulate ceramic foam with one or more metal oxides of chromium, cobalt, nickel or the like. The foam structure provides large surface area and high gas flow rates.

Next the second-stage reactor 15 accepts the syngas and converts it into a mixture of higher chain hydrocarbon molecules (preferably C₅+) the majority of which are liquid at ambient air temperature. The preferred process is a F-T process using a process intensified micro channel reactor. Process intensification technology for the F-T process is described in U.S. Pat. No. 6,211,255 (Schanke), U.S. Pat. No. 6,262,131 (Arcuri) and U.S. patent application 20,020,010,087 (Zhou), which are incorporated herein. Schanke describes a high mass-flow-rate solid-body catalyst with longitudinal promotor-lined reaction channels and transverse coolant channels. Arcuri describes a stationary catalyst with a high voidness ratio (and a concomitant high surface area) and high active metal concentration. Zhou teaches using a skeletal iron catalyst coated with active metal promotor powder which has advantageous surface area and selectivity characteristics and which may be used in either a fixed bed or a slurry F-T reactor. The effluent liquid syncrude can be stored in a tank 21 for later transport to a remote refinery, or it can be processed directly by a hydrocarbon cracking unit 17 (hydrocracker) mounted on a trailer 3 or on a pallet 5 as illustrated in FIG. 2.

The hydrocracker 17 converts the C₅+ syncrude mixture to a desired petrochemical such as diesel or gasoline. Other hydrocarbon products, such as kerosene, fuel oil, jet fuel, lubricating oil, grease, etc., may also be produced. Such hydrocrackers are commercially available. The end product fuel is stored locally in tank 23 and is dispensed by pump 25 as required.

The steam methane reforming process and the F-T process, as described above, produce byproducts which lend themselves to the portable GTL equipment. First, steam reforming produces more hydrogen than is required for the subsequent F-T process. Since reforming requires heat to raise the temperature of the feed mixture, the excess hydrogen can be used as a steady-state fuel source for the heat production. Any deficiencies or start-up requirements may be met by the source of natural gas. For example, the reforming process may use a hydrogen-fired furnace, or more preferably, an integrated catalytic combustion reactor, such as described in PCT WO 01/51194, incorporated herein. The second conducive byproduct is water produced by the F-T reaction, which because of the highly exothermic nature of the reaction, is transformed to steam. The steam byproduct supplies the steam for reforming in steady state operation, obviating the need for an external source of water. Thus, the portable GTL equipment is self-sufficient.

It is not necessary that all of the units as described above be separate modular units. Some or all of them can be combined into an integrated unit. GTL processes including single step polymerization are also within the scope of the invention.

In military applications, a source of natural gas (for example from a pipeline running across remote terrain) can be tapped as a source of fuel, easing demands on the logistical supply line.

While preferred embodiments of the invention have been illustrated in detail, it is apparent that modifications and adaptations of the preferred embodiments will occur to those skilled in the art. It is to be expressly understood that such modifications and adaptations are in the spirit and scope of the invention as set forth in the following claims:

What is claimed is:

1. An apparatus comprising,
at least one pallet (5), and
a gas-to-liquids unit (1) for transforming natural gas into hydrocarbon characterized by having a liquid phase at atmospheric pressure and ambient temperature, said gas-to-liquids unit disposed on top of said at least one pallet (5) and attached thereto.

2. The apparatus of claim 1 wherein said gas-to-liquids unit (1) further comprises,
a gas preprocessing unit (11) for filtering, desulfurizing, dehydrating, regulating pressure of, controlling flow of and mixing said natural gas with air,
a first-stage reactor (13) for converting effluent of said gas preprocessing unit into synthesis gas by a steam methane reforming reaction, and
a Fisher-Tropsch reactor (15) for polymerizing said synthesis gas to produce said liquid hydrocarbon.

3. The apparatus of claim 1 further comprising
a storage tank (21) for collecting said liquid hydrocarbon.

4. The apparatus of claim 1 further comprising
a hydrocarbon cracking unit (17) for converting said liquid hydrocarbon to a common petrochemical, disposed on said at least one pallet (5) and attached thereto.

5. The apparatus of claim 1 wherein said gas-to-liquids unit (1) is characterized by having catalyst sites which are designed and arranged for high surface-area-to-volume ratios.

6. An apparatus comprising,
at least one trailer (3), and
a gas-to-liquids unit (1) for transforming natural gas into hydrocarbon characterized by having a liquid phase at atmospheric pressure and ambient temperature, said gas-to-liquids unit disposed on top of said at least one trailer (3) and attached thereto.

7. The apparatus of claim 6 wherein said gas-to-liquids unit (1) further comprises,
a gas preprocessing unit (11) for filtering, desulfurizing, dehydrating, regulating pressure of, controlling flow of and mixing said natural gas with air,
a first-stage reactor (13) for converting effluent of said gas preprocessing unit into synthesis gas by a steam methane reforming reaction, and
a Fisher-Tropsch reactor (15) for polymerizing said synthesis gas to produce said liquid hydrocarbon.

8. The apparatus of claim 6 further comprising
a storage tank (21) for collecting said liquid hydrocarbon.

9. The apparatus of claim 6 further comprising
a hydrocarbon cracking unit (17) for converting said liquid hydrocarbon to a common petrochemical, disposed on said at least one trailer (3) and attached thereto.

10. The apparatus of claim 6 wherein said gas-to-liquids unit (1) is characterized by having catalyst sites which are designed and arranged for high surface-area-to-volume ratios.

11. A method for converting natural gas at a remote terrestrial source (7,9) to hydrocarbon characterized by having a liquid phase at atmospheric pressure and ambient temperature, comprising the steps of,
positioning a palletized or trailer-mounted gas-to-liquids unit (I) near said source (7,9),
coupling said source (7,9) to said gas-to-liquids unit (1), and
conducting natural gas thorough said gas-to-liquids unit (1).

12. The method of claim 11 wherein the step of conducting natural gas through said gas-to-liquids unit (1) comprises the steps of,
conducting said natural gas through a gas preprocessing unit (11) which filters, desulfurizes, dehydrates, regulates the pressure of, controls the flow of and mixes said natural gas with air,
conducting effluent of said gas preprocessing unit (11) through a synthesis gas reactor (13) which subjects said effluent to a steam methane reforming reaction to form synthesis gas, and
conducting said synthesis gas through a Fisher-Tropsch reactor (15) which polymerizes said synthesis gas into said hydrocarbon liquid.

13. The method of claim 11 further comprising the step of
collecting said liquid hydrocarbon.

14. The method of claim 11 further comprising the step of
cracking said liquid hydrocarbon into a common petrochemical.
